(19) United States
(12) Patent Application Publication
(10) Pub. No.: US 2025/0262142 A1
(43) Pub. Date: Aug. 21, 2025

(54) Dietary Supplement for Stimulating New Hair Growth and Reversing Hair Graying

(71) Applicant: Shylaja Hegde, Tacoma, WA (US)

(72) Inventor: Shylaja Hegde, Tacoma, WA (US)

(21) Appl. No.: 19/056,029

(22) Filed: Feb. 18, 2025

Related U.S. Application Data
(60) Provisional application No. 63/555,127, filed on Feb. 19, 2024.

(51) Int. Cl. A61K 8/67; A61K 8/66; A61K 8/96; A61K 31/375; A61K 35/02; A61K 38/44; A61Q 5/00; A61Q 7/00

(52) U.S. Cl. CPC A61K 8/676; A61K 8/66; A61K 8/965; A61K 31/375; A61K 35/02; A61K 38/44; A61Q 5/00; A61Q 7/00; C12Y 111/01006; A61K 2800/92

(57) ABSTRACT

A dietary supplement for stimulating new hair growth and for reversing the graying of hair comprising a blend from about 7.0%/wt. to about 15.0%/wt. of Vitamin C, optionally from about 50 units to about 500 units of catalase, with the balance being diatomaceous earth. This blend is formulated in powder form. Each daily dosage contains from about 2.0 grams to about 3.0 grams of the blend.

### CROSS-REFERENCE TO RELATED APPLICATIONS

This application claims priority from U.S. Provisional Application 63/555,127, filed Feb. 19, 2024.

### FIELD OF INVENTION

The present invention relates dietary supplements for stimulating new hair growth and combating or reversing hair graying. More specifically, the invention provides a formulation that is targeted to enhance the function of stem cells, particularly hair follicle stem cells (HFSCs) and melanocyte stem cells (MSCs), while protecting hair follicles from oxidative stress, thereby promoting hair growth and pigmentation.

### BACKGROUND OF THE INVENTION

In mammalian skin, hair and hair follicles are essential appendages involved in protection, thermoregulation, and sensory perception. Hair follicles are complex structures embedded within the skin, consisting of the dermal papilla, matrix, outer root sheath, and bulge. The bulge region, located in the outer root sheath, serves as the primary reservoir of the hair follicle cells. This bulk region provides a specialized microenvironment, known as the “niche” which supports the functions of these stem cells and regulates the hair growth cycle. The bulge region also contains melanocyte stem cells, which contribute to hair pigmentation. At the base of the follicle, the hair bulb houses the hair matrix, where keratinocytes proliferate and differentiate to form the hair shaft. The dermal papilla, located at the base of the follicle, provides essential signaling molecules and growth factors that promote hair growth and regulate the hair cycle. Hair growth occurs in three primary phases: anagen (growth), catagen (regression), and telogen (rest). Hair follicle stem cells exhibit activation-quiescence transitions depending on signals from their niche microenvironment, which can activate of maintain their quiescence. When certain signaling factors and activators reach a threshold, some hair follicle stem cells are prompted to enter the anagen phase, initiating hair growth.

The anagen phase is the active growth phase, lasting from about two to about seven years. During the anagen phase, hair follicle stem cells are activated by signals from the dermal papilla, proliferating and differentiating into keratinocytes, which form the hair shaft, as well as into cells that contribute to the supporting inner root sheath. Simultaneously, melanocyte stem cells differentiate into melanocyte precursors, which mature into melanocytes. These mature melanocytes migrate to the hair follicle, where they undergo melanogenesis to produce melanin, which imparts color to the hair.

The catagen phase is a short transition phase lasting about two to about three weeks, during which growth halts and the hair follicle shrinks. The hair detaches from the dermal papilla, while the hair follicle stem cells and melanocyte stem cells remain inactive in the bulge region, preserving them for the next cycle. In this phase, the dermal papilla condenses and moves upward, while the melanocytes undergo apoptosis.

The telogen phase is the resting phase, lasting about three to about four months, during which the follicle is inactive, and hair may remain in the follicle without growing. Hair shedding, referred to as exogen) may occur during the telogen phase. At the end of the telogen phase, signals from the dermal papilla reactivate hair follicle stem cells initiate a new anagen phase. Melanocyte stem cells differentiate into melanocytes, which migrate to the hair bulb to pigment the new hair shaft.

The challenges and increasing prevalence of the thinning, graying and loss of hair has become a growing concern. While genetics play a significant role in these conditions, environmental factors, lifestyle choices, diet, and commercially available hair treatments, also are key contributing factors. Studies have shown that poor dietary habits, chronic stress, lack of sleep, and exposure to environmental pollutants, such as toxins, UV radiation, and air pollution, have accelerated hair deterioration. As a result of these factors, hair thinning, loss, and graying, typically associated with older individuals, now affect younger people, even those in their twenties and thirties. These premature hair issues significantly can impact an individual's emotional and psychological well-being. Visible signs of aging, such as thinning hair and gray strands, can reduce self-esteem, increase anxiety, and promote a heightened sense of aging, regardless of age. Consequently, there is an increasing demand for solutions that address the root causes of these hair conditions, rather than simply providing temporary cosmetic fixes.

Despite growing awareness and demand for solutions for hair loss, hair thinning and hair graying, current products have limitations as they fail to effectively address the underlying causes of hair thinning, loss, and graying. For example, current topical treatments, such as minoxidil, primarily stimulate hair follicles and prolong the anagen phase. While this treatment has been effective for some individuals, it does not address the root causes of hair thinning and loss, such as poor nutrition, oxidative stress, and hormone imbalances. Additionally, its effects often are temporary, requiring continuous use, with results varying significantly among individuals.

Commercial products such as hair dyes only temporarily mask graying hair without addressing the biological causes. Further, hair dyeing may damage hair and exacerbate existing hair health issues. Such products do not provide long-term solutions for preventing or reversing the graying of hair.

There are countless nutritional supplements claiming to support hair health, but often these products target isolated factors, such as biotin for hair growth or antioxidants for protecting hair follicles. In addition, these products tend to overlook the interconnected issues of nutrition, oxidative stress, hormone regulation, and stem cell activation of hair follicles and melanocytes that are essential for long-term hair growth.

Studies have shown that nutrition plays a crucial role in regulating the function and activation of various stem cells, including hair follicles stem cells and melanocyte stem cells. Both are vital for hair growth, pigmentation, and overall skin health. The activation of these stem cells is tightly regulated by nutrient availability and metabolic signals, indicating that diet plays a key role in maintaining healthy hair follicles.

### PRIOR ART

The prior art is replete with attempts to stimulate hair growth, and to a lesser extent, to reverse the graying of hair. U.S. Pat. No. 6,103,272 to Keeney discloses topical and oral compositions for stimulating hair growth, preventing or minimizing hair loss by with colloidal silver, a mixture of herbs and vitamins extracted from plants. Keeney does not teach or suggest reversing the graying of hair. In U.S. Pat. No. 9,554,985 B2 to Noh et al. provides an agent for promoting hair growth from eight medicinal herbs but it does not suggest the reversal of graying hair. Similarly, U.S. Pat. No. 11,033,473 B2 to Okunishi et al. relates to a hair restoration/growth stimulating agent having at least one type of flavonoid Vitamin and at least one phenylpropanoid as active ingredients. The Okunishi et al. formulation does not affect or reverse the graying of hair.

U.S. Patent Application Publication 2011/0287061 A1 to Beggan is directed to a dietary composition and method for reversing gray hair, the composition including a very high concentration of catalase ranging from about 2,500 IU to about 10,000 IU as the synthetic core component along with saw palmetto, phytosterol, and nettle root extract as a 5-α reductase inhibitors. The typical recommended dosage of catalase is 250 IU to 500 IU per serving, as studies have shown higher dosages may result in adverse side effects. And, it also is well known that saw palmetto, phytosterols and nettle root extract all cause side effects, including gastrointestinal issues, sexual dysfunction, and hormonal imbalances. In addition to the known side effects, Beggan does not promote new hair growth.

Despite the efforts of the prior art, a need still exists for a comprehensive dietary supplement that effectively stimulates hair growth and reverses hair graying. Such a dietary supplement should be formulated to target the activation of stem cells, address the underlying causes of hair issues, and support long-term hair follicle health. And, such a dietary supplement should be designed for easy daily consumption alongside food and beverages, offering both convenience and practicality, while providing additional health benefits without any side effects.

### SUMMARY OF THE INVENTION

The invention of the present subject matter relates to a dietary supplement formulated to address the root causes of hair thinning, loss, and graying. The formulation combines nutritional support with anti-aging components to optimize stem cell function, nourish hair follicles, and restore pigmentation. The dietary supplement comprises a blend of a natural antioxidant, preferably ascorbic acid (Vitamin C), food-grade diatomaceous earth which provides silica and essential trace minerals for hair health, and optionally catalase, an enzyme that neutralizes oxidative stress.

Accordingly, it is an object of the present subject matter to provide a dietary supplement for stimulating new hair growth and reversing hair graying comprising ascorbic acid (Vitamin C) and food grade diatomaceous earth.

It is another object of the present subject matter to provide a dietary supplement for stimulating new hair growth and reversing hair graying comprising about 12.5%/wt. of Vitamin C, and the balance being about 87.5% of food grade diatomaceous earth.

It is yet another object of the present subject matter to provide a dietary supplement for stimulating new hair growth and reversing hair graying comprising ascorbic acid (Vitamin C), catalase, and food grade diatomaceous earth.

It is still object of the present subject matter to provide a dietary supplement for stimulating new hair growth and reversing hair graying comprising about 12.5%/wt. of Vitamin C, about 50 to about 500 units catalase, and the balance being about 87.5% of food grade diatomaceous earth.

It is a further object of the present subject matter to provide a dietary composition that promotes hair growth in humans comprising ascorbic acid (Vitamin C), food-grade diatomaceous earth and optionally catalase,

It is still a further object of the present subject matter to provide a dietary composition comprising ascorbic acid (Vitamin C), catalase, and food-grade diatomaceous earth.

It is yet a further object of the present subject matter to provide a dietary supplement for promoting hair growth in individuals with alopecia comprising ascorbic acid (Vitamin C), food-grade diatomaceous earth, and optionally catalase.

It is a yet another object of the present subject matter to provide a dietary composition that improves skin, bone, teeth, and nail health (via collagen support), and supports gut health, comprising ascorbic acid (Vitamin A), catalase and food-grade diatomaceous earth.

It is an additional object of the present subject matter to provide a dietary supplement for stimulating new hair growth and reversing hair graying which restores gray hair to its natural color.

It is another object of the present subject matter to provide a dietary supplement for stimulation new hair growth and reversing hair graying, which increases hair growth and improves overall hair health.

It is a further object of the present subject matter to provide a dietary supplement for stimulating new hair growth and reversing hair graying, which does not produce any potential side effects associate with its use.

These and other objects of the present invention are accomplished by providing a dietary supplement for stimulating new hair growth and reversing hair graying comprising Vitamin C, food-grade diatomaceous earth, and optionally catalase.

### DETAILED DESCRIPTION OF THE INVENTION

In the following description, the terms “supplement”, “dietary supplement”, “nutritional supplement”, “dietary composition”, “dietary formulation”, “formulation”, “composition”, derivates thereof and similar terms may be used throughout the specification and it is to be understood that such terms are interchangeable and all refer to the dietary supplement for stimulating new hair growth and reversing hair graying of the present subject matter.

Also, it is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

The present subject matter is directed a potent combination of ingredients designed to stimulate hair follicle stem cells and melanocyte stem cells, thereby promoting hair growth, regeneration, and pigmentation. Emerging research has demonstrated that specific nutrients can activate stem cells and support tissue regeneration across various tissues. For example, studies have indicated that nutrient-rich foods can stimulate stem cell activity in cardiomyocytes, neural cells, and promote heart muscle cell regeneration due to its flavonoid content. Green tea, rich in polyphenols such as epigallocatechin gallate, has been shown to stimulate neural stem cells and neurogenesis. Pomegranate is believed to enhance stem cell proliferation, particularly in bone marrow, while berries, high in antioxidants and polyphenols, reduce oxidative stress and support stem cell proliferation. These findings highlight the potential of specific nutrients to activate stem cells and promote tissue regeneration, including the regeneration of hair follicles.

Food-grade diatomaceous earth is a key ingredient of the dietary supplement of the present subject matter. It is rich in 94% silica and contains fifteen (15) essential trace minerals, including iron, copper, and zinc, all of which are critical for supporting the health of hair, skin, and bones. Studies suggest that the silica in diatomaceous earth promotes hair follicle regeneration through several mechanisms. First, through collagen production. Collagen is essential for maintaining the structural integrity of hair follicles and the surrounding skin, providing and optimal environment for hair follicle stem cell function. Second, through activation of hair follicle stem cells. Preliminary studies suggest that silica nanoparticles can stimulate stem cell proliferation and differentiation, potentially influencing hair growth cycles.

Another potent ingredient of the dietary supplement of the present subject matter is ascorbic acid (Vitamin C), which enhances collagen synthesis, protects hair follicles from oxidative damage, and improves nutrient absorption, contributing to vibrant and healthy hair.

In one embodiment, another key ingredient is catalase. Catalase is a powerful antioxidant, shields hair from oxidative stress and helps maintain natural hair color by protecting melanocytes from damage.

Accordingly, in one embodiment, the dietary supplement for stimulating new hair growth and reversing hair graying comprises ascorbic acid (Vitamin C) and food-grade diatomaceous earth. Preferably, the dietary supplement comprises from between about 7.0% by weight to about 15.0% by weight of Vitamin C, with the balance being food-grade diatomaceous earth. More preferably, the formulation comprises about 12.5% by weight of Vitamin C and about 87.5% by weight of food-grade diatomaceous earth.

In another embodiment, the dietary supplement for stimulating new hair growth and reversing hair graying comprises about between about 7.0% by weight to about 15.0% by weight of Vitamin C, from between about 50 units to about 500 units of catalase, with the balance being food-grade diatomaceous earth. More preferably, the comprises about 12.5% by weight of Vitamin C, about 250 units catalase, and 87.5% by weight of food-grade diatomaceous earth.

The dietary supplement of the present subject matter carefully is formulated with these synergistic ingredients, which work together to stimulate both hair growth and melanin synthesis. The dietary supplement is provided in powder form. In one embodiment, the daily dosage contains from about 2.0 grams to about 3.0 grams of this blend of ingredients. In a preferred embodiment, one dosage contains about 2.5 grams of the blend.

The dietary supplement of the present subject matter has been tested on several subjects as described hereinbelow.

Subject 1 (47-year-old female). Initially, this subject had about 30 to about 35% visible gray hair and a receding hairline. The subject consumed a daily dosage of 2.5 grams of the dietary supplement for eighteen (18) months. After three weeks, she reported increased energy levels, improved hair volume, smoothness, and shine, as well as improvements in nail and bone health. By week 12, the subject no longer needed to color her hair as most of the white hairs had darkened and some appeared more gray. After six (6) months, the subject reported additional improvements in teeth, gums, skin, and new hair growth. After twelve (12) months, the effects were increasing prominent with no observed side effects. Significant improvements in skin, nails, teeth, and gums were reported along with a noticeable increase in new hair growth.

Subject 2 (48-year-old female). This subject experienced noticeable hair loss, especially in the frontal area. The subject consumed the daily dosage of 2.5 grams of the dietary supplement for five (5) months. After two (2) months, the subject reported new hair growth. By the fourth (4^{th}) month, the subject's hair grew rapidly, covering the areas previously affected by hair loss. In addition, hair growth was observed on her eyebrows and eyelashes. The overall health of her hair improved, appearing softer and darker.

Subject 3 (53-year-old female). The subject consumed a daily dosage of 2.5 grams of the dietary supplement for three (3) months. Within the first month, the subject reported improved bone health, with stronger bones and no cracking sounds. After three months, significant new hair growth was observed along her receding hairline. The subject also reported improvements in skin and hair color, with white hairs becoming more gray, with several shades of gray visible.

Subject 4 (54-year-old female). The subject consumed a daily dosage of 2.5 grams of the dietary supplement for three (3) months. After three (3) months, new hair growth was observed along her receding hairline. The subject's skin appeared healthier and more youthful, and her hair color became darker, with her white hair darkening noticeably.

Subject 5 (56-year-old female). The subject consumed a daily dosage of 2.5 grams of the dietary supplement for four (4) months. After two (2) months, most of the subject's white hair had turned black. Her hair also appeared darker, softer, and shinier. The subject no longer needed to color her hair every ten days and began coloring it once a month. As the subject continued to consume the dietary supplement, the frequency of the hair coloring further decreased.

While individual responses varied, the dietary supplement of the present subject matter demonstrated efficacy in stimulating hair growth, restoring hair color, and providing collagen-related anti-aging benefits, including improvements in skin health, bone health, as well as enhancements in the condition of nails, teeth, and gums. These effects have been observed over several months of continued and consistent use. Although full reversal of gray hair was not observed in all individuals, factors such as age, genetic predisposition, and overall health, can influence the results. Notwithstanding the above comments, improvements in hair color, pigmentation, and overall hair health was observed in subjects with three to six months of continued and consistent use of the dietary supplement. Given the natural hair growth cycle, which spans up to six years, it is hypothesized that, upon completion of the full hair cycle, the hair follicles could be replenished with stimulated hair follicle stem cells and melanocyte stem cells, potentially reversing all gray hair in healthy individuals under 65 years of age.

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, under the provisions of 35 U.S.C § 112, sixth paragraph, unless the element is expressly recited using the phrase “means for . . . ” and no method or process step herein is to be construed under those provisions unless the step, or steps, are expressly recited using the phrase “step(s) for . . . ”

1. A dietary supplement for stimulating hair growth and reversing the graying of the hair comprising a blend of Vitamin C and food-grade diatomaceous earth.

2. The dietary supplement for stimulating hair growth and reversing the graying of the hair in accordance with claim 1, comprising from between about 7.0% by weight to about 15.0% by weight of Vitamin C with the balance being food-grade diatomaceous earth.

3. The dietary supplement for stimulating hair growth and reversing the graying of the hair in accordance with claim 2, comprising from about 12.5% by weight Vitamin C and 87.5% of food-grade diatomaceous earth.

4. The dietary supplement for stimulating hair growth and reversing the graying of the hair in accordance with claim 1, further comprising catalase.

5. The dietary supplement for stimulating hair growth and reversing the graying of the hair in accordance with claim 4, comprising from between about 7.0% by weight to about 15.0% by weight of Vitamin C, from between about 50 units to about 500 units catalase, with the balance being food-grade diatomaceous earth.

6. The dietary supplement for stimulating hair growth and reversing the graying of the hair in accordance with claim 5, comprising from about 12.5% by weigh of Vitamin C, from 250 units catalase, with the balance being food-grade diatomaceous earth.

7. The dietary supplement for stimulating hair growth and reversing the graying of the hair in accordance with claim 1, wherein said dietary supplement is in powder form containing from about 2.0 grams to about 3.0 grams of the said blend per daily dosage.

8. The dietary supplement for stimulating hair growth and reversing the graying of the hair in accordance with claim 1, wherein said dietary supplement is in powder form containing about 2.5 grams of said blend per daily dosage.

9. A dietary supplement for stimulating hair growth and reversing the graying of the hair consisting of a blend of Vitamin C and food-grade diatomaceous earth.

10. The dietary supplement for stimulating hair growth and reversing the graying of the hair in accordance with claim 1, consisting of from between about 7.0% by weight to about 15.0% by weight of Vitamin C with the balance being food-grade diatomaceous earth.

11. The dietary supplement for stimulating hair growth and reversing the graying of the hair in accordance with claim 10, consisting of from about 12.5% by weight Vitamin C and 87.5% of food-grade diatomaceous earth.

12. The dietary supplement for stimulating hair growth and reversing the graying of the hair in accordance with claim 9, wherein said dietary supplement is in powder form containing from about 2.0 grams to about 3.0 grams of the said blend per daily dosage.

13. The dietary supplement for stimulating hair growth and reversing the graying of the hair in accordance with claim 12, wherein said dietary supplement is in powder form containing about 2.5 grams of said blend per daily dosage.

14. A dietary supplement for stimulating hair growth and reversing the graying of the hair consisting of a blend of Vitamin C, catalase, and food-grade diatomaceous earth.

15. A dietary supplement for stimulating hair growth and reversing the graying of the hair in accordance with claim 14, consisting of a blend of from between about 7.0% by weight to about 15.0% by weight of Vitamin C, from between about 50 units to about 500 units catalase, with the balance being food-grade diatomaceous earth.

16. The dietary supplement for stimulating hair growth and reversing the graying of the hair in accordance with claim 15, consisting of about 12.5% by weigh of Vitamin C, from 250 units catalase, with the balance being food-grade diatomaceous earth.

17. The dietary supplement for stimulating hair growth and reversing the graying of the hair in accordance with claim 14, wherein said dietary supplement is in powder form containing from about 2.0 grams to about 3.0 grams of the said blend per daily dosage.

18. The dietary supplement for stimulating hair growth and reversing the graying of the hair in accordance with claim 17, wherein said dietary supplement is in powder form containing about 2.5 grams of said blend per daily dosage.
